(19) United States
(12) Patent Application Publication
(10) Pub. No.: US 2025/0268740 A1
(43) Pub. Date: Aug. 28, 2025

(54) CERVICAL IMMOBILIZATION DEVICE

(71) Applicant: Pinnacle Devices, LLC, Rochester, NY (US)

(72) Inventor: Michael BEINETTI, Rochester, NY (US)

(73) Assignee: Pinnacle Devices, LLC, Rochester, NY (US)

(21) Appl. No.: 19/062,679

(22) Filed: Feb. 25, 2025

Related U.S. Application Data
(60) Provisional application No. 63/557,801, filed on Feb. 26, 2024.

(51) Int. Cl. A61F 5/055

(52) U.S. Cl. CPC A61F 5/055

(57) ABSTRACT

A cervical immobilization device (CID) includes a front collar. The front collar includes a chest brace configured to extend downward from both shoulders of a patient and rest against a chest of the patient. An adjustable headband is configured to strap around a forehead of the patient. First and second lateral head supports are configured to attach and extend vertically downward from the headband. The first and second lateral head supports are configured to form a rigid connection to the front collar. First and second underarm straps each have first and second ends. The first ends are configured to attach to opposing sides of the chest brace below the patient's shoulders. The second ends are configured to attach to opposing sides of the CID above the patient's shoulders when the CID is worn by the patient.

### CROSS REFERENCE TO RELATED APPLICATIONS

This application is a non-provisional application of, and claims the benefit of the filing date of, U.S. provisional application 63/557,801, filed Feb. 26, 2024, entitled “CERVICAL IMMOBILIZATION DEVICE,” the contents of which are incorporated herein by reference in their entirety.

### TECHNICAL FIELD

The present disclosure relates to cervical immobilization devices. More specifically, the disclosure relates to cervical immobilization devices that include a front collar with a chest brace, wherein a pair of underarm straps attach to the chest brace below the patient's shoulders and attach to the front collar above the patient's shoulders.

### BACKGROUND

Cervical immobilization devices (CIDs), or cervical neck braces, are used to substantially immobilize an injured patient in the field. However, prior art CIDs often have multiple components that are difficult to attach to a patient, especially if the patient is unconscious. Furthermore, prior art CIDs do not adequately immobilize the patient even when properly applied. Moreover, such prior art CIDs are often uncomfortable for the patient to wear.

Additionally, prior art CIDs often have chin bracing components which are positioned around the chin area to further immobilize the head. Problematically however, such chin bracing components make it difficult for a first responder to intubate a patient when needed, because they are not easily removable and substantially impair chin movement. As a result, prior art CIDs that brace the chin often have to be completely removed when a patient is being intubated, which unnecessarily heightens the risk of further injury to the patient.

Accordingly, there is a need for a cervical immobilization device which is more comfortable to wear, is more easily attached and adjusted in the field, and better immobilizes the head of a patient than prior art CIDs. Additionally, there is a need for a cervical immobilization device which enables a patient to be intubated without having to remove substantially all of the CID.

### BRIEF DESCRIPTION OF THE INVENTION

The present disclosure offers advantages and alternatives over the prior art by providing a cervical immobilization device (CID), which is easily attachable and adjustable in the field and is comfortable to wear. The CID includes a front collar with a chest brace that is rigidly connected to a headband to immobilize forward movement of the head.

Additionally, adjustable first and second underarm straps are connected at their distal ends directly to the chest brace below the patient's shoulder and directly to the front collar above the patient's shoulder. The first and second underarm straps are configured to extend under and abut against the floor of the armpit so that, when tightened snug against the patient's armpits, the underarm straps prevent the head of the patient from moving rearward.

Moreover, the CID of the present disclosure may include a chin brace that is removably attached to the front collar. The chin brace is configured to be the sole component of the CID required to be removed from the CID to enable a patient to be intubated while wearing the CID.

A cervical immobilization device in accordance with one or more aspects of the present disclosure includes a front collar. The front collar includes a chest brace configured to extend downward from both shoulders of a patient and rest against a chest of the patient. An adjustable headband is configured to strap around a forehead of the patient. First and second lateral head supports are configured to attach and extend vertically downward from the headband. The first and second lateral head supports are configured to form a rigid connection to the front collar. First and second underarm straps each have first and second ends. The first ends are configured to attach to opposing sides of the chest brace below the patient's shoulders. The second ends are configured to attach to opposing sides of the CID above the patient's shoulders when the CID is worn by the patient.

Another cervical immobilization device in accordance with one or more aspects of the present disclosure includes a front collar. The front collar includes a chest brace configured to extend downward from both shoulders of a patient and rest against a chest of the patient. An adjustable headband is configured to strap around a forehead of the patient. First and second lateral head supports are configured to attach and extend vertically downward from the headband. The first and second lateral head supports are configured to form a rigid connection to the front collar. A chin brace is removably attached to opposing sides of the front collar. The chin brace is configured to provide additional support to immobilize the patient, when the CID is being worn by the patient. The chin brace is configured to be the sole component of the CID required to be removed from the CID to enable the patient to be intubated while wearing the CID.

Another cervical immobilization device in accordance with one or more aspects of the present disclosure includes a front collar. The front collar includes a single integral structure, which includes the structural features of a chest brace, first and second shoulder pieces and first and second collar projections. The chest brace is configured to extend downward from both shoulders of a patient and rest against a chest of the patient. The first and second shoulder pieces are integrally attached to opposing upper ends of the chest brace. The first and second shoulder pieces are configured to project over and rest upon the shoulders of the patient to support the front collar thereon. The first and second lateral collar projections are integrally attached to opposing upper ends of the chest brace. The first and second lateral collar projections extend vertically upwards therefrom. An adjustable headband is configured to strap around a forehead of the patient. First and second lateral head supports are configured to attach and extend vertically downward from the headband. The first and second lateral head supports are configured to adjustably and rigidly attach to the first and second lateral collar projections to adjust a distance between the headband and the front collar. First and second underarm straps each having first and second ends. The first end of the first underarm strap is configured to attach to a left side of the chest brace below the patient's shoulder and the second end of the first underarm strap is configured to attach to a right side of the CID above the patient's shoulder. The first end of the second underarm strap is configured to attach to a right side of the chest brace below the patient's shoulder and the second end of the second underarm strap is configured to attach to a left side of the CID above the patient's shoulder.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits and advantages described herein.

### BRIEF DESCRIPTION OF THE DRAWINGS

The disclosure will be more fully understood from the following detailed description taken in conjunction with the accompanying drawings, in which:

FIG. 1 depicts an example of a front perspective view of a cervical immobilization device, according to aspects described herein;

FIG. 2 depicts an example of a rear perspective view of the cervical immobilization device of FIG. 1 being worn by a patient, according to aspects described herein;

FIG. 3 depicts an example of a perspective view of the cervical immobilization device of FIG. 1 being worn by a patient, according to aspects described herein;

FIG. 4 depicts an example of a perspective view of a front collar with a chest brace of the cervical immobilization device of FIG. 1, according to aspects described herein;

FIG. 5 depicts an example of a perspective view of a headband and first and second lateral head supports of the cervical immobilization device of FIG. 1, according to aspects described herein;

FIG. 6 depicts an example of a chin brace of the cervical immobilization device of FIG. 1, according to aspects described herein;

FIG. 7A depicts an example of a partially exploded view of the cervical immobilization device of FIG. 1 being worn by a patient with the chin brace separated from the cervical immobilization device, according to aspects described herein; and

FIG. 7B depicts an example of a perspective view of the cervical immobilization device of FIG. 1 being worn by a patient without a chin brace while being intubated, according to aspects described herein.

### DETAILED DESCRIPTION OF THE INVENTION

Certain examples will now be described to provide an overall understanding of the principles of the structure, function, manufacture, and use of the methods, systems, and devices disclosed herein. One or more examples are illustrated in the accompanying drawings. Those skilled in the art will understand that the methods, systems, and devices specifically described herein and illustrated in the accompanying drawings are non-limiting examples and that the scope of the present disclosure is defined solely by the claims. The features illustrated or described in connection with one example may be combined with the features of other examples. Such modifications and variations are intended to be included within the scope of the present disclosure.

The terms “significantly”, “substantially”, “approximately”, “about”, “relatively,” or other such similar terms that may be used throughout this disclosure, including the claims, are used to describe and account for small fluctuations, such as due to variations in processing from a reference or parameter. Such small fluctuations include a zero fluctuation from the reference or parameter as well. For example, they can refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

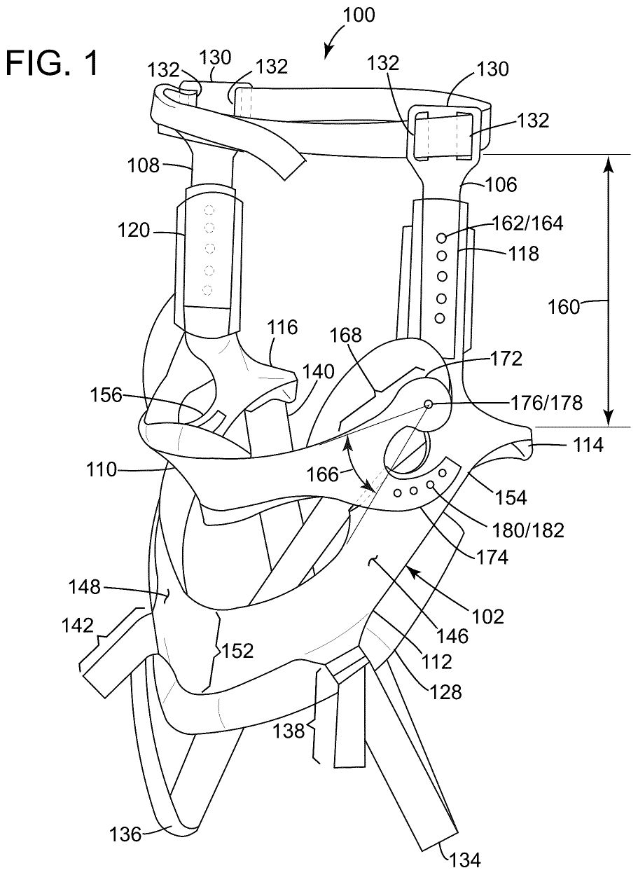

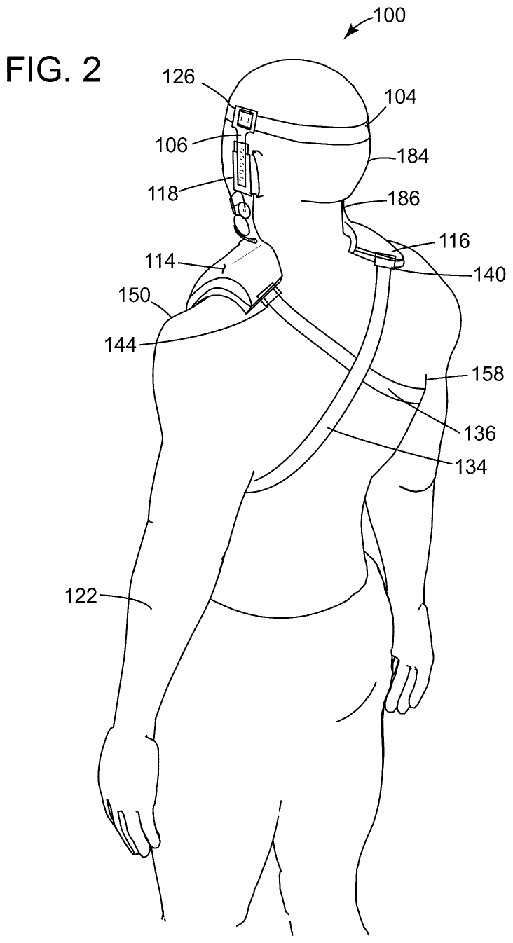

Referring to FIGS. 1 and 2, an example is depicted of a front perspective view off of a patient 122 (FIG. 1) and a rear perspective view when being worn by a patient 122 (FIG. 2) of a cervical immobilization device (CID) 100, according to aspects described herein. The CID includes a front collar 102, an adjustable headband 104, a first lateral head support 106 and a second lateral head support 108. The CID may also include a chin brace 110. The front collar 102 includes a chest brace 112, a first shoulder piece 114, a second shoulder piece 116, a first lateral collar projection 118 and a second lateral collar projection 120.

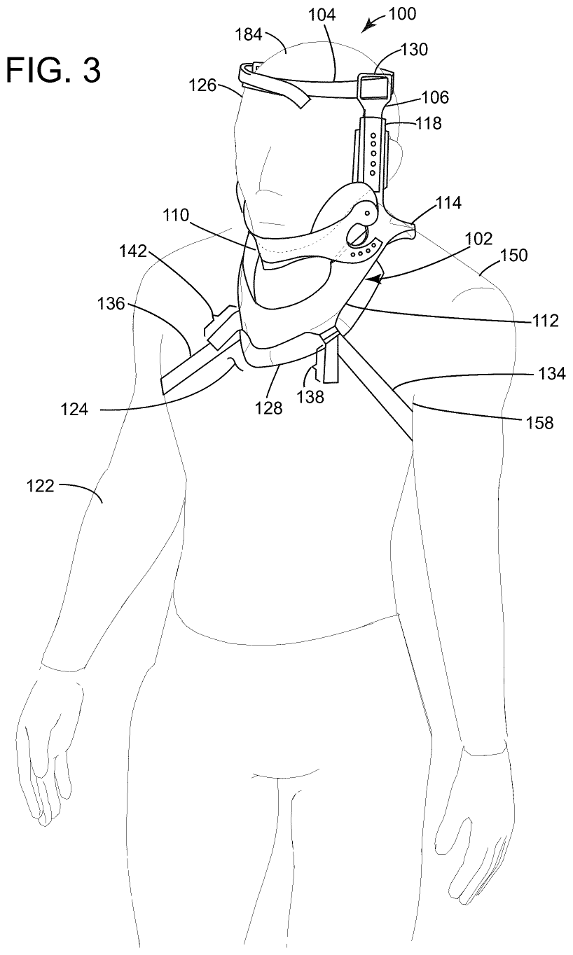

As mentioned above, the front collar 102 of the CID 100 includes the chest brace 112. The chest brace is configured to extend downward from both shoulders of a patient 122 (see FIG. 3) and rest against a chest 124 of the patient 122. The chest brace 112 may be made of a hard plastic material of any other appropriately rigid material. The chest brace 112 may also have padding 128 disposed thereon for the comfort of the patient.

The adjustable headband 104 of the CID 100 is configured to strap around a forehead 126 of the patient 122. The headband is illustrated herein as utilizing hook and loop fasteners to adjustably fasten to the forehead 126 of the patient 122. However, any number of appropriate fastening systems may be used. For example, buckles, snaps, tabs or other fastening devices may be used to enable adjustment to the headband around a patient's head.

The first and second lateral head supports 106, 108 are configured to attach and extend vertically downward from the headband 104. The lateral head supports may be made of a hard plastic or any appropriate rigid material. As illustrated, the lateral head supports 106, 108 each have a head section 130, with two parallel headband slots 132 for the headband 104 to feed through and attach thereto. However, the lateral head supports 106, 108 may be attached to the headband 104 via any number of appropriate attachment systems. For example, buckles, snaps, tabs or other fastening devices may be used. Additionally, the lateral head supports 106, 108 may be integrally or permanently attached to the headband 104.

The first and second lateral head supports 106, 108 are configured to form a rigid connection to the front collar 102. As will be discussed in greater detail herein, the rigid connection illustrated in FIGS. 1 and 2 is the direct connection between the first and second lateral head supports 106, 108 and the associated first and second lateral collar projections 118, 120 of the front collar 102. However, the lateral head supports 106, 108 may be rigidly connected to the collar 102 by any number of appropriate connections either directly or indirectly. For example, the lateral head supports 106, 108 may be rigidly attached to a chin brace, similar to the chin brace 110. The chin brace may then be rigidly connected to the chest brace 112 of the collar 102 to provide an indirect rigid connection between the lateral head supports 106, 108 and the collar 102.

The CID 100 also includes a first underarm strap 134 and a second underarm strap 136. The first underarm strap 134 has a first end 138 and a second end 140. The second underarm strap 136 also has a first end 142 and a second end 144. The first ends 138, 142 of each underarm strap 134, 136 are configured to attach to a left side 146 and an opposing right side 148 of the chest brace 112 below the patient's shoulders 150. The first ends 138, 142 may also connect to a lower end portion 152 of the chest brace 112.

The underarm straps 134, 136 may be removably attached to the CID via any number of appropriate connection devices. For example, the underarm straps 134, 136 may be attachable at either end using quick slide and release buckles as illustrated in FIGS. 1 and 2. Alternatively, the underarm straps 134, 136 may be removable and/or attachable utilizing a hook and loop fastener system. Moreover, the underarm straps 134, 136 may be removably attachable at only one end and permanently attached at the other end (as illustrated in FIGS. 1 and 2), or the underarm straps 134, 136 may be removably attachable at both ends.

The second ends 140, 144 of each underarm strap 134, 136 are configured to attach to opposing sides of the CID 100 above the patient's shoulders 150 when the CID 100 is worn by the patient 122 (see FIG. 3). For example, the second ends 140, 144, may be attached a left side and a right side of the collar 102 of the CID 100 above the shoulders 150 of the patient 122. More specifically, the second ends 140, 144 may be attached to an left upper end 154 and an opposing right upper end 156 of the chest brace 112 of the front collar 102.

Though the underarm straps 134, 136 are illustrated herein as being attached to the front collar 102 of the CID 100 above the patient's shoulders 150, it is within the scope of the present disclosure that the underarm straps 134, 136 may also attach to other components of the CID 100. For example, in an alternative embodiment, the lateral head supports 106, 108 of the CID 100 may be configured to attach to the second ends 140, 142 of the underarm straps 134, 136.

The first and second underarm straps 134, 136 may be configured to extend under and abut against a floor of the patient's armpits 158 when the CID 100 is worn by the patient 122 (see FIG. 3). Armpit, as used herein, means the hollow beneath the junction of the arm and shoulder of a patient 122. More specifically, the armpit includes the pyramid shaped part of the body between the upper arm and the side of the thorax of a patient 122. The armpit is floored by the skin of the armpit.

The first and second underarm straps 134, 136, may extend from the front of the patient 122 to the rear of the patient without crisscrossing in the back of the patient 122. However, for added stability, the underarm straps 134, 136 may be made to crisscross at the back of the patient 122. More specifically, the first end 138 of the first underarm strap 134 may be configured to attach to the left side 146 of the chest brace 112 below the patient's shoulder 150 and the second end 140 of the first underarm strap 134 may be configured to attach to the right side of the CID 100 above the patient's shoulder 150. Additionally, the first end 142 of the second underarm strap 136 may be configured to attach to a right side 148 of the chest brace 112 below the patient's shoulder 150 and the second end 144 of the second underarm strap 136 may be configured to attach to a left side of the CID 100 above the patient's shoulder 150.

The front collar 102 may also include the first and second shoulder pieces 114, 116. The first and second shoulder pieces 114, 116 are integrally attached to the left upper end 154 of the chest brace 112 and the opposing right upper end 156 of the chest brace 112 of the front collar 102. The first and second shoulder pieces 114, 116 are configured to project over and rest upon the shoulders 150 of the patient 122 to support the front collar 102 thereon. The first and second shoulder pieces 114, 116 provide a positive stop for the front collar 102, while the front collar 102 advantageously provides support for the positioning of the other components of the CID 100 (e.g., the first and second lateral supports 106, 108, the headband 104 and the chin brace 110).

The front collar 102 may also include the first and second lateral collar projections 118, 120. The first and second lateral collar projections 118, 120 are also integrally attached to opposing upper ends 154, 156 of the chest brace 112. The first and second lateral collar projections 118, 120 extend vertically upwards from their associated upper ends 154, 156 of the chest brace 112. The first and second lateral collar projections 118, 120 are configured to provide a rigid but adjustable attachment to the first and second lateral head supports 106, 108. The adjustable attachment of the lateral collar projections 118, 120 to the lateral head supports 106, 108 enables a distance 160 between the headband 104 and the front collar 102 to be adjusted. More specifically, the lateral collar projections 118, 120 may adjust a distance 160 between the headband 104 and the first and second shoulder pieces 116, 118 of the front collar 102. The adjustability of the distance 160 enables the CID 100 to accommodate various neck lengths and head sizes of the patient 122.

The lateral collar projections 118, 120 may be adjustably attached to the lateral head supports 106, 108 by any number of appropriate attachment systems. For example, by a hook and loop attachment system, or by utilizing a system of buckles.

However, the attachment system illustrated in FIGS. 1 and 2 utilizes a system of adjustment tabs 162 positioned on the lateral head supports 106, 108 and adjustment thru-holes 164 positioned on the lateral collar projections 118, 120. More specifically, a plurality of headband adjustment tabs 162 may be positioned vertically on the first and second lateral head supports 106, 108. Additionally, a plurality of headband adjustment thru-holes 164 may be positioned vertically on the first and second lateral collar projections 118, 120. The headband adjustment thru-holes 164 are sized to receive the headband adjustment tabs 162. During operation, one or more of the plurality of headband adjustment tabs 162 mate with (or press fit into) one or more of the plurality of headband adjustment thru-holes 164 to provide a rigid, but adjustable, connection between the first and second lateral head supports 106, 108 and the first and second lateral collar projections 118, 120 respectively. The adjustable connection enables the adjustment of the distance 160 between the headband 104 and the shoulder projections 114, 116 of the front collar 102.

Alternatively, the attachment system may also utilize a system of adjustment tabs 162 positioned on the lateral collar projections 118, 120 and adjustment thru-holes 164 positioned on the lateral head supports 106, 108. More specifically, a plurality of headband adjustment tabs 162 may be positioned vertically on the first and second lateral collar projections 118, 120. Additionally, a plurality of headband adjustment thru-holes 164 may be positioned vertically on the first and second head supports 106, 108. The operation of this embodiment of an attachment system with the adjustment tabs 162 positioned on the lateral collar projections 118, 120 remains essentially the same as the embodiment of the attachment system with the adjustable tabs 162 positioned on the lateral head supports 106, 108.

The CID may also include the chin brace 110. The chin brace 110 is removably attached to opposing sides of the front collar 102. The chin brace 110 is configured to provide additional support to immobilize the patient 122, when the CID 100 is being worn by the patient 102. Advantageously, the chin brace 110 is configured to be the sole component of the CID 100 that is required to be removed from the CID 100 to enable the patient 122 to be intubated or perform other medically necessary interventions while still wearing the CID 100.

As illustrated in FIGS. 1 and 2, the chin brace 110 is pivotally and adjustably attached to opposing sides of the front collar 102 to enable a pivot angle 166 between the chin brace 110 and the front collar 102 to be adjusted. More specifically, the pivot angle 166 may be between the chin brace 110 and the chest brace 112 of the front collar 102.

The chin brace 110 may be made removably attachable and/or pivotably attachable to the front collar 102 through any number of appropriate attachment systems. However, in the chin brace 110 illustrated in FIGS. 1 and 2, the attachment system of the chin brace 110 includes a first forked chin brace end portion 168 and a second forked chin brace end portion 170. Each forked chin brace end portion 168, 170 includes a first pivoting chin brace extension 172 pivotally and removably attached to the front collar 102. Additionally, each forked chin brace end portion 168, 170 includes a second adjusting chin brace extension 174 adjustably and removably attached to the front collar 102.

More specifically, each first pivoting chin brace extension 172 includes one of a first tab 176 or a first thru-hole 178, which is configured to mate with the other of the first tab 176 or the first thru-hole 178 positioned on the front collar 102. In other words, if the first tab 176 is positioned on the first pivoting chin brace extension 172, then the first thru-hole 178 will be positioned on the front collar 102. Alternatively, if the first tab 176 is positioned on the front collar 102, then the first thru-hole 178 will be positioned on the first pivoting chin brace extension 172. In either case, the first tab 176 is configured to pivot within the first thru-hole 178 to enable the chin brace 110 to pivot relative to the front collar 102.

Additionally, each second adjusting chin brace extension 174 includes one of a plurality second tabs 180 or a plurality of second thru-holes 182, which are configured to mate with the other of the plurality of second tabs 180 or the plurality of second thru-holes 182 positioned on the front collar 102. In other words, if the plurality of second tabs 180 are positioned on the second adjusting chin brace extension 174, then the plurality of second thru-holes 182 will be positioned on the front collar 102. Alternatively, if the plurality of second tabs 180 are positioned on the front collar 102, then the plurality of second thru-holes 182 will be positioned on the second adjusting chin brace extension 174. In either case, one or more of the plurality of second tabs 180 mate with one or more of the plurality of second thru-holes 182 to provide an adjustable connection between the second adjusting chin brace extensions 174 and the front collar 102 to enable adjustment of the pivot angle 166 between the chin brace 110 and the front collar 102.

Referring to FIG. 3, an example is depicted of a perspective view of the cervical immobilization device 100 being worn by the patient 122, according to aspects described herein. During operation, the CID 100 may be easily fit to the patient 122 by placing the front collar 102 over the patient's chest 124 and shoulders 150. The first and second underarm straps 134, 136 are wrapped around the patient 122 and may be made to crisscross in the back of the patient 122. The first ends 138, 142 of the underarm straps 134, 136 are connected at the lower end portion 152 of the chest brace 112 in front of the patient 122 and below the shoulders 150. The second ends 140, 144 of the underarm straps 134, 136 are connected to the front collar 102 in the rear of the patient 122 and above the shoulders 150. The underarm straps 134, 136 may then be tightened snug against the patient's armpits 158. The headband 104 may then be wrapped around the forehead 126 of the patient 122 and the lateral head supports 106, 108 may be connected to the lateral collar projections 118, 120 to provide a rigid connection between the headband 104 and front collar 102.

Advantageously, even without the chin brace 110 connected to the CID 100, the patient's neck 186 and head 184 are substantially immobilized at this point for several reasons. More specifically, the first and second shoulder pieces 114, 116 provide a positive stop against the patient's shoulders 150 in the downward direction, while the first and second underarm straps 134, 136 provide a positive stop against the patient's armpits 158 in the upwards direction. Therefore, once the rigid connection between the lateral head supports 106, 108 and the lateral collar projections 118, 120 is made, the distance 160 between the headband 104 and shoulder pieces 114, 116 of the collar 102 is fixed.

Additionally, the chest brace 112 of the collar 102 is firmly anchored to the chest 124 of the patient 122 by the underarm straps 134, 136 to prevent forward movement of the head 184 of the patient 122. Also, the anchored chest brace 112 combined with the positive stop of the shoulder pieces 116, 118 against the shoulders 150 prevent rearward movement of the head 184 of the patient 122. Moreover, the crisscrossed underarm straps 134, 136 substantially restrict rotational movement of the head 184 of the patient 122.

For added support, the chin brace 110 may be removably attached to the front collar 102 to further immobilize the head 184 and neck 186 of the patient. The chin brace 110 may be pivoted at its first pivoting chin brace extensions 172 to comfortably abut against the chin of the patient 122 and then may be locked rigidly in place by the second adjusting chin brace extensions 174. However, the chin brace 110 is the sole component of the CID 100 that is required to be removed from the CID 100, in order to enable the patient 122 to be intubated. Advantageously, the CID, even with the chin brace 110 removed, will securely immobilize the patient 122 during the intubation process.

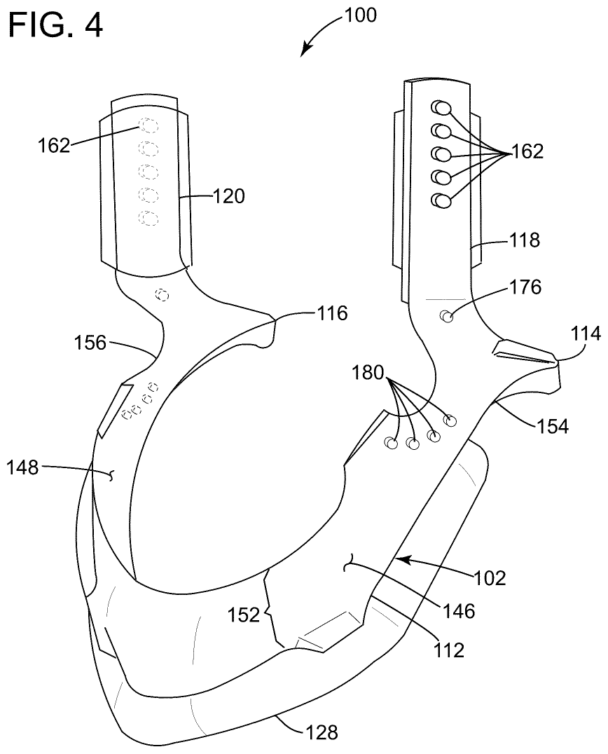

Referring to FIG. 4, an example is depicted of a perspective view of the front collar 102, which includes the chest brace 112 of the cervical immobilization device 100 of FIG. 1, according to aspects described herein. The collar 102 is the main support component of the CID 100.

The collar 102 provides several advantageous structural features for immobilizing and securing the head 184 and neck 186 of the patient 122 in a single integral structure. Those structural features include at least the following:
the chest brace 112 of the collar 102, for providing the leverage needed to prevent forward and rearward movement of the head 184 of the patient 122;
the first and second shoulder pieces 114, 116 of the collar 102, for providing a positive stop of the collar 102 against the shoulders 150 of the patient 122; and
the first and second lateral collar projections 118, 120 of the collar 102, for providing a rigid connection to the first and second lateral head supports 106, 108 and ultimately a rigid connection to the headband 104 for securing the head 184 of the patient 122.

The chest brace 112 of the collar 102 is designed to rest against the chest 124 of a patient 122 and provide the leverage needed to prevent forward and rearward movement of the head 184 of the patient 122. The chest brace 112 utilizes the chest 124 as a positive stop against any forward movement of the head 184 of the patient 122. Additionally, when the chest brace 112 is anchored to the chest 124 of the patient 122 by the underarm straps 134, 136, the chest brace 112 substantially prevents any rearward movement of the head 184 of the patient 122.

The first and second shoulder pieces 114, 116 of the collar 102 provide the positive stop against the shoulders 150 of the patient 122. Additionally, the shoulder pieces 114, 116 further restrict rearward movement of the patient's head 184.

The first and second lateral collar projections 118, 120 of the collar 102 are designed to adjustably mate with the lateral head supports 106, 108 to provide the required rigid connection between the collar 102 and the headband 104. Accordingly, once the collar 102 is anchored to the patient 122 by the underarm straps 134, 136 and the headband 104 is rigidly connected to the collar 102 via the lateral head supports 106, 108, the head 184 and neck 186 of the patient 122 are restrained.

The lateral collar projections 118, 120 are illustrated in FIG. 4 as having the adjustment tabs 162 disposed thereon, wherein they would mate with the adjustment thru-holes 164 on the lateral head supports 106, 108. Alternatively, however, the lateral collar projections 118, 120 may also have the adjustment thru-holes 164 disposed thereon, wherein they would mate with the adjustment tabs 162 disposed on the lateral head supports 106, 108.

The front collar 102 is also illustrated in FIG. 4 as having the first tabs 176 and second tabs 180 disposed thereon. As such, the first tabs 176 would mate with the first thru-holes 178 disposed on the first pivoting chin brace extension 172 and the second tabs 180 would mate with the second thru-holes 182 disposed on the second adjusting chin brace extension 174 of the chin brace 110. Alternatively, however, The front collar 102 may have the first and second thru-holes 178, 182 disposed thereon. Accordingly, the first thru-holes 178 would mate with the first tabs 176 disposed on the first pivoting chin brace extension 172 and the second thru-holes 182 would mate with the second tabs 180 disposed on the second adjusting chin brace extension 174 of the chin brace 110.

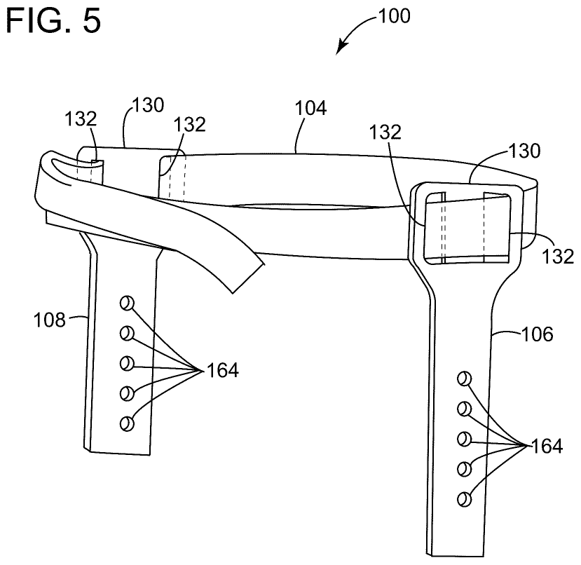

Referring to FIG. 5, an example is depicted of a perspective view of the headband 104 and first and second lateral head supports 106, 108 of the cervical immobilization device 100 of FIG. 1, according to aspects described herein.

The headband 104, as illustrated, wraps around a patient's forehead 126 and is adjustable via a hook and loop attachment system. However, any number of other attachment systems may also be used. For example, buckles, snaps, tabs or other fastening devices may be used to secure the headband 104 to the forehead 126 of the patient 122.

The first and second lateral head supports 106, 108 may be composed of a hard plastic material or any other appropriately rigid material that can adequately provide a rigid connection with the lateral collar projections 118, 120 of the collar 102.

As illustrated, the headband 104 feeds through the headband slots 132 on the head section 130 of the lateral head supports 106, 108. However, the lateral head supports 106 may be attached via any number of other appropriate attachment systems, such as via hook and loop, buckles, snaps, tabs or the like. Additionally, the lateral head supports 106, 108 may be integrally and permanently attached to the headband 104.

The lateral head supports 106, 108 are illustrated as being adjustably attachable to the lateral collar projections 118, 120 of the collar 102 via a plurality of adjustment tabs 162. The adjustment tabs are configured to mate to a corresponding plurality of adjustment thru-holes 164. More specifically, a plurality of headband adjustment tabs 162 may be positioned vertically on the first and second lateral head supports 106, 108. Additionally, a plurality of headband adjustment thru-holes 164 may be positioned vertically on the first and second lateral collar projections 118, 120. The headband adjustment thru-holes 164 are sized to receive the headband adjustment tabs 162. During operation, one or more of the plurality of headband adjustment tabs 162 mate with (or press fit into) one or more of the plurality of headband adjustment thru-holes 164 to provide a rigid, but adjustable, connection between the first and second lateral head supports 106, 108 and the first and second lateral collar projections 118, 120 respectively. The adjustable connection enables the adjustment of the distance 160 between the headband 104 and the shoulder projections 114, 116 of the front collar 102. Alternatively, the plurality of headband adjustment tabs 162 may be positioned vertically on the first and second lateral collar projections 118, 120 and the plurality of headband adjustment thru-holes 164 may be positioned vertically on the first and second head supports 106, 108.

The lateral head supports 106, 108 are illustrated in FIG. 5 as having the adjustment tabs 162, wherein they would mate with the adjustment thru-holes 164 on the lateral head supports 106, 108. Alternatively, however, the lateral collar projections 118, 120 may also have the adjustment thru-holes 164 disposed thereon, wherein they would mate with the adjustment tabs 162 disposed on the lateral head supports 106, 108.

Though the lateral head supports 106, 108 are illustrated as being adjustably attachable to the lateral collar projections 118, 120 of the collar 102 via a plurality of adjustment tabs 162, the lateral head supports 106, 108 may be adjustably attached to the lateral collar projections 118, 120 via any number of other attachment systems. For example, via hook and loop fasteners, buckles or the like.

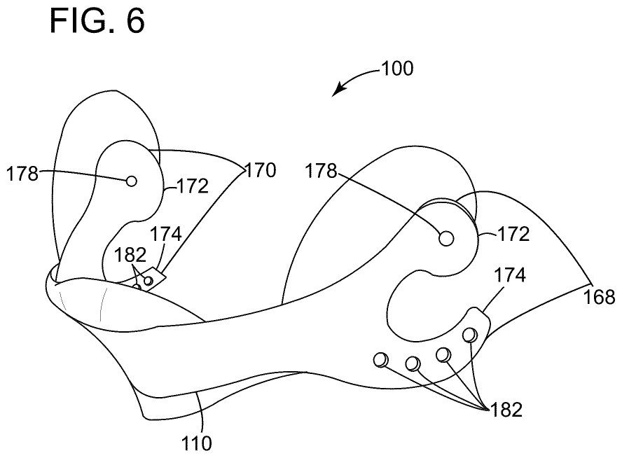

Referring to FIG. 6, an example is depicted of the chin brace 110 of the cervical immobilization device 100 of FIG. 1, according to aspects described herein. As discussed earlier herein, the chin brace 110 provides additional support to the head 184 of the patient 122 when attached to the CID 100. The chin brace 110 may be made of a hard plastic material or other appropriate material that can provide a rigid brace against the chin of the patient 122. The chin brace 110 may also include padding 128 to provide a more comfortable fit for the patient 122.

Advantageously, the chin brace 110 is configured to be the sole component of the CID 100 that is required to be removed from the CID 100 to enable the patient 122 to be intubated while still wearing the CID 100.

The chin brace 110 may be made removably attachable and/or pivotably attachable to the front collar 102 through any number of appropriate attachment systems. However, in the chin brace 110 illustrated herein, the attachment system of the chin brace 110 includes a first forked chin brace end portion 168 and a second forked chin brace end portion 170. Each forked chin brace end portion 168, 170 includes a first pivoting chin brace extension 172 pivotally and removably attached to the front collar 102. Additionally, each forked chin brace end portion 168, 170 includes a second adjusting chin brace extension 174 adjustably and removably attached to the front collar 102.

More specifically, each first pivoting chin brace extension 172 includes one of a first tab 176 or a first thru-hole 178, which is configured to mate with the other of the first tab 176 or the first thru-hole 178 positioned on the front collar 102. In other words, if the first tab 176 is positioned on the first pivoting chin brace extension 172, then the first thru-hole 178 will be positioned on the front collar 102. Alternatively, if the first tab 176 is positioned on the front collar 102, then the first thru-hole 178 will be positioned on the first pivoting chin brace extension 172. In either case, the first tab 176 is configured to pivot within the first thru-hole 178 to enable the chin brace 110 to pivot relative to the front collar 102.

Additionally, each second adjusting chin brace extension 174 includes one of a plurality second tabs 180 or a plurality of second thru-holes 182, which are configured to mate with the other of the plurality of second tabs 180 or the plurality of second thru-holes 182 positioned on the front collar 102. In other words, if the plurality of second tabs 180 are positioned on the second adjusting chin brace extension 174, then the plurality of second thru-holes 182 will be positioned on the front collar 102. Alternatively, if the plurality of second tabs 180 are positioned on the front collar 102, then the plurality of second thru-holes 182 will be positioned on the second adjusting chin brace extension 174. In either case, one or more of the plurality of second tabs 180 mate with one or more of the plurality of second thru-holes 182 to provide an adjustable connection between the second adjusting chin brace extensions 174 and the front collar 102 to enable adjustment of the pivot angle 166 between the chin brace 110 and the front collar 102.

The chin brace 110 is illustrated in FIG. 6 as having the first thru-holes 178 disposed on the first pivoting chin brace extension 172 and the second thru-holes 182 disposed on the second adjusting chin brace extension 174. As such, the first and second thru-holes 178, 182 would mate with the first and second tabs 176, 180 respectively, wherein the first and second tabs 176, 180 would be mounted on the front collar 102. Alternatively, however, the front collar 102 may have the first and second thru-holes 178, 182 mounted thereon. Accordingly, the first and second tabs 176, 180 would be mounted on the chin brace 110 and would mate with the first and second thru-holes 178, 182 mounted on the front collar 102.

In an alternative embodiment, the chin brace 110 may be a chin strap (not shown). The chin strap may be removably attached to the collar 102 by any number of appropriate fastening systems, such as hook and loop fasteners, snaps, tabs or the like.

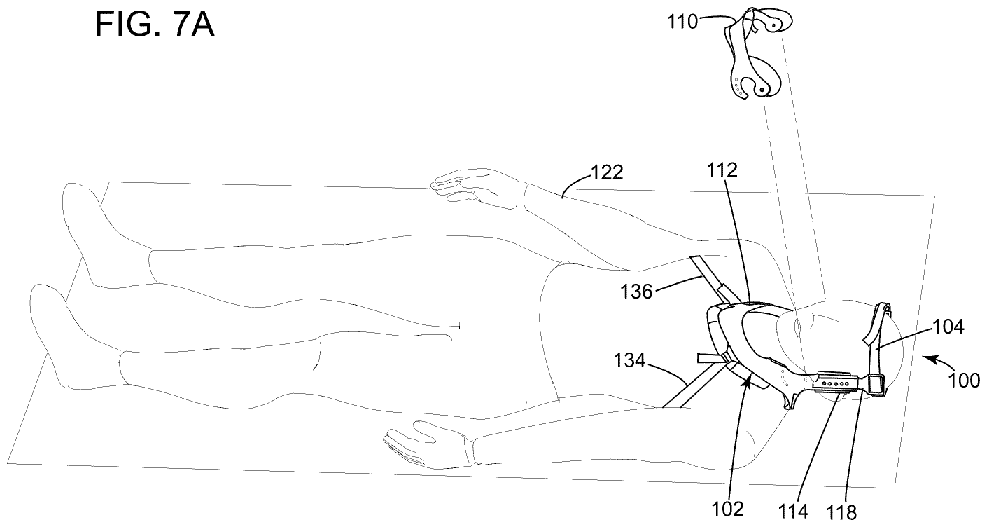

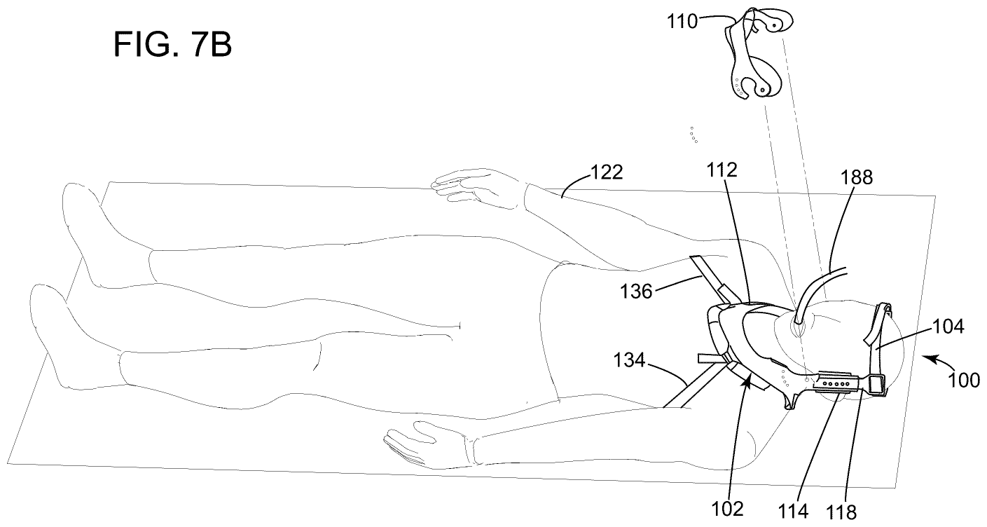

Referring to FIG. 7A, an example is depicted of a partially exploded view of the cervical immobilization device 100 being worn by the patient 122 with the chin brace 110 separated from the CID 100, according to aspects described herein.

The chin brace 110 may be easily removed from the patient 122 in order to provide access to the mouth of the patient 122 and to enable the patient to be intubated. In the embodiments illustrated herein, the chin brace 110 may be removed by pulling on the first pivoting chin brace extensions 172 and the second adjusting chin brace extensions 174 to separate the first and second tabs 176, 180 from the first and second thru-holes 178, 182 respectively.

Referring to FIG. 7B, an example is depicted of a perspective view of the CID 100 being worn by the patient 122 without a chin brace 110 while being intubated with an intubation tube 188, according to aspects described herein.

The chin brace 110 is the sole component of the CID 100 that is required to be removed from the CID 100, in order to enable the patient 122 to be intubated. Advantageously, the CID, even with the chin brace 110 removed, will securely immobilize the patient 122 during the intubation process, wherein the patient 122 may be intubated with an intubation tube 188.

More specifically, even without the chin brace 110 attached to the CID 100, the first and second shoulder pieces 114, 116 provide a positive stop against the patient's shoulders 150 in the downward direction, while the first and second underarm straps 134, 136 provide a positive stop against the patient's armpits 158 in the upwards direction. Therefore, once the rigid connection between the lateral head supports 106, 108 and the lateral collar projections 118, 120 is made, the distance 160 between the headband 104 and shoulder pieces 114, 116 of the collar 102 is fixed.

Additionally, independent of the chest brace 110 being connected to the collar 102, the chest brace 112 of the collar 102 is firmly anchored to the chest 124 of the patient 122 by the underarm straps 134, 136 to prevent forward movement of the head 184 of the patient 122. Also, the anchored chest brace 112 combined with the positive stop of the shoulder pieces 116, 118 against the shoulders 150 prevent rearward movement of the head 184 of the patient 122. Moreover, the crisscrossed underarm straps 134, 136 substantially restrict rotational movement of the head 184 of the patient 122.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

Although the invention has been described by reference to specific examples, it should be understood that numerous changes may be made within the spirit and scope of the inventive concepts described. Accordingly, it is intended that the disclosure not be limited to the described examples, but that it have the full scope defined by the language of the following claims.

What is claimed is:

1. A cervical immobilization device (CID), comprising:
a front collar comprising:
a chest brace configured to extend downward from both shoulders of a patient and rest against a chest of the patient;
an adjustable headband configured to strap around a forehead of the patient;
first and second lateral head supports configured to attach and extend vertically downward from the headband, the first and second lateral head supports configured to form a rigid connection to the front collar; and
first and second underarm straps each having first and second ends, wherein:
the first ends are configured to attach to opposing sides of the chest brace below the patient's shoulders, and
the second ends are configured to attach to opposing sides of the CID above the patient's shoulders when the CID is worn by the patient.

2. The cervical immobilization device of claim 1, wherein the second ends of the first and second underarm straps are configured to attach to the front collar.

3. The cervical immobilization device of claim 1, wherein the first and second underarm straps are configured to extend under and abut against a floor of the patient's armpits when the CID is worn by the patient.

4. The cervical immobilization device of claim 1, wherein:
the first end of the first underarm strap is configured to attach to a left side of the chest brace and the second end of the first underarm strap is configured to attach to a right side of the CID; and
the first end of the second underarm strap is configured to attach to a right side of the chest brace and the second end of the second underarm strap is configured to attach to a left side of the CID.

5. The cervical immobilization device of claim 1, wherein the front collar further comprises:
first and second shoulder pieces integrally attached to opposing upper ends of the chest brace, the first and second shoulder pieces configured to project over and rest upon the shoulders of the patient to support the front collar thereon.

6. The cervical immobilization device of claim 1, wherein the front collar further comprises:
first and second lateral collar projections integrally attached to opposing upper ends of the chest brace, the first and second lateral collar projections extending vertically upwards therefrom;
wherein the first and second lateral collar projections are configured to adjustably attached to the first and second lateral head supports to adjust a distance between the headband and the front collar.

7. The cervical immobilization device of claim 6, comprising:
a plurality of headband adjustment tabs positioned vertically on the first and second lateral head supports; and
a plurality of headband adjustment thru-holes positioned vertically on the first and second lateral collar projections, wherein the headband adjustment thru-holes are sized to receive the headband adjustment tabs;
wherein one or more of the plurality of headband adjustment tabs mate with one or more of the plurality of headband adjustment thru-holes to provide an adjustable connection between the first and second lateral head supports and the first and second lateral collar projections respectively to enable the adjustment of the distance between the headband and the front collar.

8. The cervical immobilization device of claim 6, comprising:
a plurality of headband adjustment tabs positioned vertically on the first and second lateral collar projections; and
a plurality of headband adjustment thru-holes positioned vertically on the first and second lateral head supports, wherein the headband adjustment thru-holes are sized to receive the headband adjustment tabs;
wherein one or more of the plurality of headband adjustment tabs mate with one or more of the plurality of headband adjustment thru-holes to provide an adjustable connection between the first and second lateral head supports and the first and second lateral collar projections respectively to enable the adjustment of the distance between the headband and the front collar.

9. The cervical immobilization device of claim 1, comprising:
a chin brace removably attached to opposing sides of the front collar;
wherein the chin brace is configured to provide additional support to immobilize the patient, when the CID is being worn by the patient; and
wherein the chin brace is configured to be the sole component of the CID required to be removed from the CID to enable the patient to be intubated while wearing the CID.

10. The cervical immobilization device of claim 9, wherein the chin brace is pivotally and adjustably attached to opposing sides of the front collar, to enable a pivot angle between the chin brace and the front collar to be adjusted.

11. The cervical immobilization device of claim 10, wherein the chin brace further comprises first and second forked chin brace end portions, each forked chin brace end portion comprising:
a first pivoting chin brace extension pivotally and removably attached to the front collar; and
a second adjusting chin brace extension adjustably and removably attached to the front collar.

12. The cervical immobilization device of claim 11, wherein:
each first pivoting chin brace extension includes one of a first tab or a first thru-hole, which is configured to mate with the other of the first tab or the first thru-hole positioned on the front collar, wherein the first tab is configured to pivot within the first thru-hole to enable the chin brace to pivot relative to the front collar; and
each second adjusting chin brace extension includes one of a plurality second tabs or a plurality of second thru-holes, which are configured to mate with the other of the plurality of second tabs or the plurality of second thru-holes positioned on the front collar, wherein one or more of the plurality of second tabs mate with one or more of the plurality of second thru-holes to provide an adjustable connection between the second adjusting chin brace extensions and the front collar to enable adjustment of the pivot angle between the chin brace and the front collar.

13. A cervical immobilization device (CID), comprising:
a front collar comprising:
a chest brace configured to extend downward from both shoulders of a patient and rest against a chest of the patient;
an adjustable headband configured to strap around a forehead of the patient;
first and second lateral head supports configured to attach and extend vertically downward from the headband, the first and second lateral head supports configured to form a rigid connection to the front collar; and
a chin brace removably attached to opposing sides of the front collar;
wherein the chin brace is configured to provide additional support to immobilize the patient, when the CID is being worn by the patient; and
wherein the chin brace is configured to be the sole component of the CID required to be removed from the CID to enable the patient to be intubated while wearing the CID.

14. The cervical immobilization device of claim 13, wherein the chin brace is pivotally and adjustably attached to opposing sides of the front collar, to enable a pivot angle between the chin brace and the front collar to be adjusted.

15. The cervical immobilization device of claim 14, wherein the chin brace further comprises first and second forked chin brace end portions, each forked chin brace end portion comprising:
a first pivot chin brace extension pivotally and removably attached to the front collar; and
a second adjusting chin brace extension adjustably and removably attached to the front collar.

16. The cervical immobilization device of claim 13, comprising:
first and second underarm straps each having first and second ends, wherein:
the first ends are configured to attach to opposing sides of the chest brace below the patient's shoulders,
the second ends are configured to attach to opposing sides of the CID above the patient's shoulders, and
the first and second underarm straps are configured to extend under and abut against a floor of the patient's armpits when the CID is worn by the patient.

17. The cervical immobilization device of claim 16, wherein:
the first end of the first underarm strap is configured to attach to a left side of the chest brace and the second end of the first underarm strap is configured to attach to a right side of the CID; and
the first end of the second underarm strap is configured to attach to a right side of the chest brace and the second end of the second underarm strap is configured to attach to a left side of the CID.

18. The cervical immobilization device of claim 13, wherein the front collar further comprises:
first and second shoulder pieces integrally attached to opposing upper ends of the chest brace, the first and second shoulder pieces configured to project over and rest upon the shoulders of the patient to support the front collar thereon.

19. A cervical immobilization device (CID), comprising:
a front collar comprising a single integral structure including the structural features of:
a chest brace configured to extend downward from both shoulders of a patient and rest against a chest of the patient,
first and second shoulder pieces integrally attached to opposing upper ends of the chest brace, the first and second shoulder pieces configured to project over and rest upon the shoulders of the patient to support the front collar thereon, and
first and second lateral collar projections integrally attached to opposing upper ends of the chest brace, the first and second lateral collar projections extending vertically upwards therefrom;
an adjustable headband configured to strap around a forehead of the patient;
first and second lateral head supports configured to attach and extend vertically downward from the headband, the first and second lateral head supports configured to adjustably and rigidly attach to the first and second lateral collar projections to adjust a distance between the headband and the front collar;
first and second underarm straps each having first and second ends, wherein:
the first end of the first underarm strap is configured to attach to a left side of the chest brace below the patient's shoulder and the second end of the first underarm strap is configured to attach to a right side of the CID above the patient's shoulder; and
the first end of the second underarm strap is configured to attach to a right side of the chest brace below the patient's shoulder and the second end of the second underarm strap is configured to attach to a left side of the CID above the patient's shoulder.

20. The cervical immobilization device of claim 19, comprising:
a chin brace removably attached to opposing sides of the front collar;
wherein the chin brace is configured to provide additional support to immobilize the patient, when the CID is being worn by the patient; and
wherein the chin brace is configured to be the sole component of the CID required to be removed from the CID to enable the patient to be intubated while wearing the CID.
